(19) United States
(12) Patent Application Publication
(10) Pub. No.: US 2025/0208083 A1
(43) Pub. Date: Jun. 26, 2025

(54) ELECTRODE DEVICE, WATER QUALITY MEASURING DEVICE AND WATER QUALITY MEASURING METHOD

(71) Applicant: HORIBA Advanced Techno, Co., Ltd., Kyoto-shi (JP)

(72) Inventors: Kensuke TODA, Kyoto-shi (JP); Yuichiro KOMATSU, Kyoto-shi (JP)

(21) Appl. No.: 18/985,321

(22) Filed: Dec. 18, 2024

(30) Foreign Application Priority Data
Dec. 26, 2023 (JP) 2023-219118

(51) Int. Cl. G01N 27/30; G01N 33/18

(52) U.S. Cl. CPC G01N 27/301; G01N 33/18

(57) ABSTRACT

An electrode device comprises a reference electrode, the reference electrode comprises a reference internal electrode that contains a first component, a reference housing within which the reference internal electrode is housed, a reference internal solution that is housed within the reference housing and within which the reference internal electrode is immersed, and a dissolving material that contains the first component and that is arranged in solid form within the reference internal solution.

### CROSS REFERENCE TO RELATED APPLICATIONS

This application claims the benefit of priority of Japanese application no. 2023-219118, filed on Dec. 26, 2023, which is incorporated herein by reference.

### BACKGROUND OF THE INVENTION

### Field of the Invention

The present application relates to an electrode device, a water quality measuring device and a water quality measuring method.

### Description of the Related Art

Conventionally, an electrode device might, for example, comprise a reference electrode, and the reference electrode might comprise a reference internal electrode; a reference housing within which the reference internal electrode is housed; and a reference internal solution that is housed within the reference housing and within which the reference internal electrode is immersed (e.g., JP2017-215313A). It so happens, where the reference internal electrode contains a component which decomposes when immersed in a liquid-like substance, that there is a possibility that an increase in the temperature of the reference internal solution may cause the solubility of said component in the reference internal solution to become large, as a result of which the reference internal electrode may be eluted into the reference internal solution.

### SUMMARY OF THE INVENTION

The problem is therefore to provide an electrode device, a water quality measuring device and a water quality measuring method that will make it possible to suppress elution of a reference internal electrode into a reference internal solution.

There is provided an electrode device comprising a reference electrode,
the reference electrode comprises
   a reference internal electrode that contains a first component;
   a reference housing within which the reference internal electrode is housed;
   a reference internal solution that is housed within the reference housing and within which the reference internal electrode is immersed; and
   a dissolving material that contains the first component and that is arranged in solid form within the reference internal solution.

There is provided a water quality measuring device comprising the electrode device.

There is provided a water quality measuring method wherein the electrode device is used to measure a water quality value of a target liquid.

### BRIEF DESCRIPTION OF DRAWINGS

FIG. 1 is a schematic diagram of a water quality measuring device associated with an embodiment.

FIG. 2 is a perspective view showing the principal components of an electrode device associated with same embodiment, a portion of which has been rendered in see-through fashion to reveal the interior.

FIG. 3 is a bottom view of an electrode device associated with same embodiment.

FIG. 4 is a sectional view of the principal components in a section taken along IV-IV in FIG. 3.

FIG. 5 is a sectional view of the principal components in a section taken along V-V in FIG. 3.

FIG. 6 is an enlarged view of region VI in FIG. 5.

### DETAILED DESCRIPTION OF THE INVENTION

So as to, for example, facilitate understanding, dimensions of constituent elements at the respective drawings may be shown larger or smaller than their actual dimensions; furthermore, dimensional ratios are not necessarily consistent from drawing to drawing. Moreover, so as to, for example, facilitate understanding, constituent elements may be shown at the respective drawings in partially omitted fashion.

Where terminology including first, second, or other such ordinal numbers is used, this is done to describe a diverse variety of constituent elements; as such terminology is employed only for the purpose of distinguishing one constituent element from other constituent element(s), it should be understood that such terminology constitutes no particular limitation with respect to the constituent elements. Note, furthermore, with respect to the number of constituent element(s) which include ordinal number(s) that are present, there is no particular limitation with respect thereto, it being possible for there to be, for example, one thereof. Furthermore, ordinal number(s) employed below in the specification and drawings may differ from ordinal number(s) recited in the claims.

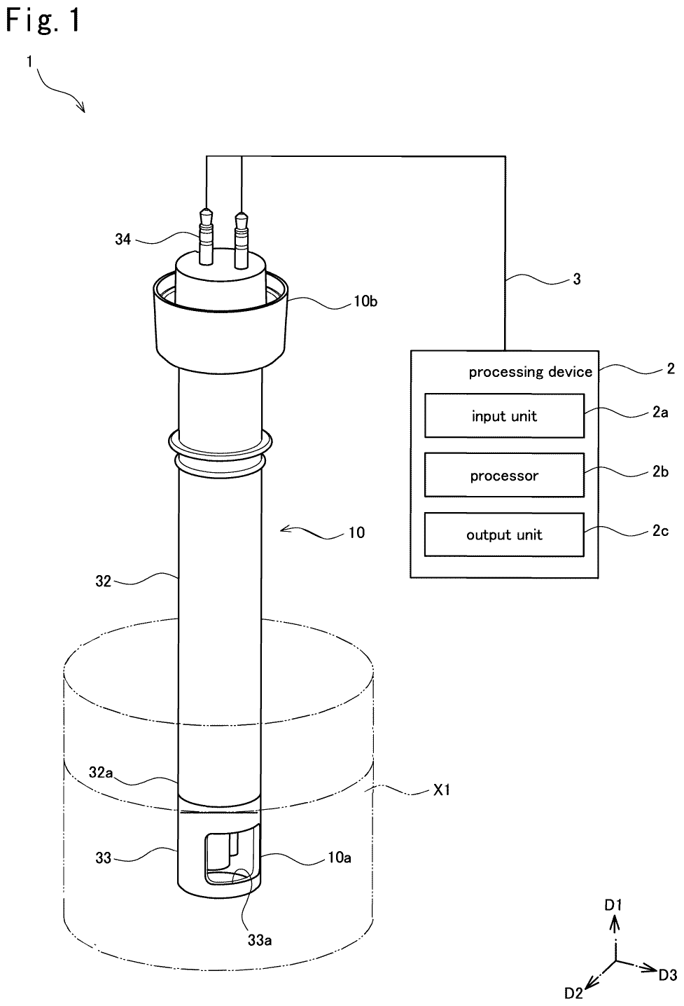

Below, embodiments of an electrode device and a water quality measuring device are described with reference to FIG. 1 through FIG. 6. Note that the embodiments below are exemplary, being intended to assist in understanding the constitutions and so forth of the electrode devices and the water quality measuring devices, and should not be interpreted in such a way as to limit the constitutions of the electrode devices and the water quality measuring devices.

As shown in FIG. 1, water quality measuring device 1 comprises electrode device 10 which detects water quality of a target liquid X1 that is a target for measurement. Furthermore, water quality measuring device 1 may, e.g., as is the case in the present embodiment, comprise processing device 2 which is capable of communication with electrode device 10; and communication means 3 which is capable of causing electrode device 10 and processing device 2 to be in communication.

Processing device 2 may, e.g., as is the case in the present embodiment, comprise input unit 2a at which information is input; processor 2b which processes information; and output unit 2c at which information is output. While there is no particular limitation with respect thereto, processing device 2 may be a mobile terminal (e.g., a smart device, tablet computer, notebook-type personal computer, or the like).

Communication means 3 might, for example, be wired communication means (e.g., a communication cable, a wired LAN, etc.); or it might, for example, be wireless communication means (e.g., Wi-Fi, a wireless LAN, etc.). Moreover, electrode device 10 and processing device 2 might, for example, be constituted such that they are separate; or, they might, for example, be constituted such that they are separably connected; or, they might, for example, be constituted such that they are inseparably integral.

While there is no particular limitation with respect thereto, input unit 2a may,, for example, be button(s), touch panel(s), keyboard(s), mouse(s), and/or the like. In addition, information in the form of an instruction to begin measurement, information in the form of measurement conditions, and/or the like might, for example, be input at input unit 2a.

Processor 2b might, for example, comprise a CPU, MPU, and/or other such processor(s); ROM, RAM, and/or other such memory or memories; various interfaces, and so forth. More specifically, processor 2b might, for example, comprise an acquisition unit which acquires information from electrode device 10, input unit 2a, and/or the like; a storage unit which information; an arithmetic unit which performs arithmetic operations with respect to information (e.g., water quality values); and a control unit which controls various unit(s) (e.g., electrode device 10 and/or output unit 2c) of water quality measuring device 1.

While there is no particular limitation with respect thereto, output unit 2c might, for example, be a display unit (e.g., a monitor or an indicator lamp) that displays information, a sound-emitting unit (e.g., a speaker or a buzzer) that produces information in the form of sound, a signal output unit that outputs a signal to the exterior (e.g., a central monitoring panel or the like), and/or the like. In addition, output unit 2c might, for example, output information that has been measured (e.g., water quality values) and/or the like.

As shown in FIG. 1 through FIG. 3, electrode device 10 comprises measurement electrode 11 and reference electrode 21 for measuring particular water quality value(s) of target liquid X1. As a result, it is possible, for example, by causing electrode device 10 to detect the difference in electric potential between measurement electrode 11 and reference electrode 21, and causing processing device 2 to calculate a water quality value based on said difference in electric potential, to measure a particular water quality value of target liquid X1. Note that electrode device 10 comprising measurement electrode 11 and reference electrode 21 might, for example, also be called a composite electrode.

While there is no particular limitation with respect thereto, water quality item(s) of target liquid X1 detected by electrode device 10 might, for example, be pH, oxidation-reduction potential (ORP), particular ion(s), and/or the like. Moreover, while there is no particular limitation with respect thereto, said particular ion(s) might, for example, be ammonium ion, nitrate ion, potassium ion, chloride ion, calcium ion, sodium ion, and/or the like.

Furthermore, electrode device 10 may, e.g., as is the case in the present embodiment, be formed so as to be elongated in first direction D1. In addition, it is also possible, for example, to adopt a constitution in which a particular water quality value is detected by electrode device 10 as a result of having caused first end 10a in first direction D1 of electrode device 10 to be immersed in target liquid X1.

Moreover, while there is no particular limitation with respect thereto, electrode device 10 might, for example, be grasped by a jig or by a person who wishes to carry out measurement, and a particular water quality value of target liquid X1 which has been placed within a container might be detected. Furthermore, electrode device 10 might, for example, be attached to a frame or the like, and detection might be carried out with respect to particular water quality value(s) of target liquid X1 in the form of treated sewage (e.g., aerobic tank water, anaerobic tank water, incoming effluent tank water), treated human excreta, wastewater from semiconductor manufacturing plants, wastewater from chemical product manufacturing plants, wastewater from food manufacturing plants, water drawn from rivers and reservoirs, well water and groundwater, aquafarm holding pond and wastewater, plant factory liquid fertilizer and wastewater therefrom, and so forth.

Furthermore, electrode device 10 may, e.g., as is the case in the present embodiment, comprise temperature sensor 31, the end of which is exposed, for detecting the temperature of target liquid X1. Moreover, electrode device 10 may, for example, comprise other water quality sensor(s) (e.g., dissolved oxygen sensor, electrical conductivity sensor, turbidity sensor, etc.) for detecting other water quality item(s) dissolved oxygen, electrical (e. g., conductivity, turbidity, etc.).

Furthermore, electrode device 10 may, e.g., as is the case

in the present embodiment, comprise case 32 which extends cylindrically in first direction D1; cover 33 which covers the end of temperature sensor 31; and terminal 34 which is electrically connected to the exterior. While there is no particular limitation with respect thereto, in accordance with the present embodiment, cover 33 is arranged at first end 10a in first direction D1 of electrode device 10, and terminal 34 is arranged at second end 10b in first direction D1 of electrode device 10.

While there is no particular limitation with respect thereto, case 32 and cover 33 might, for example, be formed from metal, hard resin, glass, or the like, and might have rigidity so as not to be subject to deformation. Furthermore, cover 33 may, e.g., as is the case in the present embodiment, comprise opening 33a for entry thereinto by target liquid X1. Moreover, cover 33 may, e.g., as is the case in the present embodiment, be connected to first end 32a in first direction D1 of case 32.

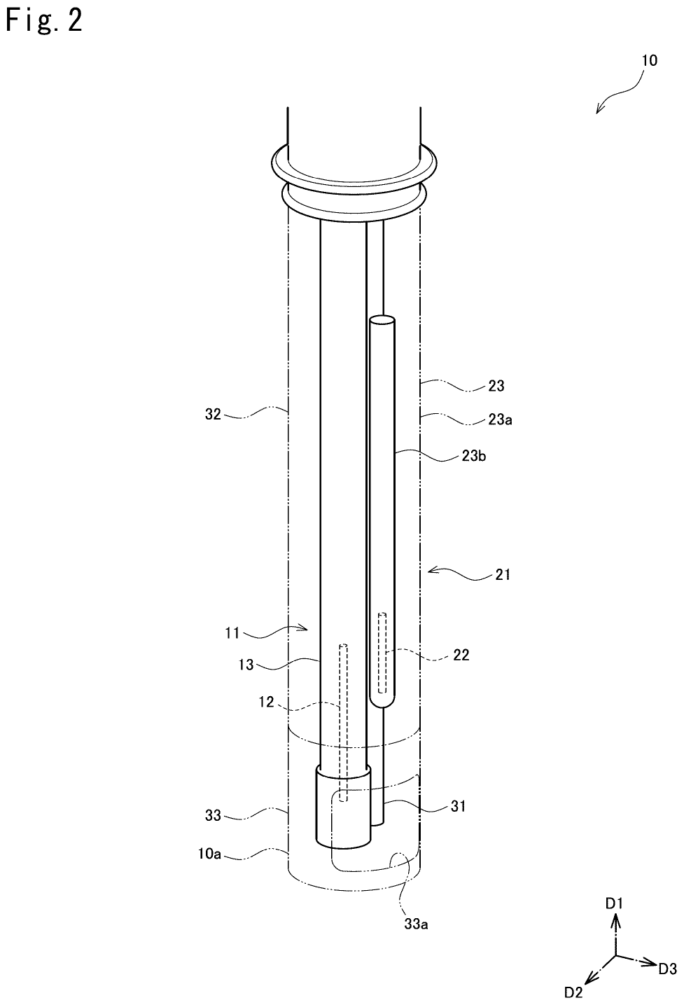

As shown in FIG. 2 through FIG. 4, measurement electrode 11 comprises measurement internal electrode 12; measurement housing 13 within which measurement internal electrode 12 is housed; and measurement internal solution 14 which is housed within measurement housing 13 and within which measurement internal electrode 12 is immersed. Furthermore, measurement electrode 11 may, e.g., as is the case in the present embodiment, comprise responsive membrane 15 which produces an electric potential in response to the concentration of a particular water quality item (e.g., hydrogen ion or a particular ion) of target liquid X1; and lead wire(s) 16 that electrically connect measurement internal electrode 12 which has electrical conductivity and terminal 34 (see FIG. 1).

While there is no particular limitation with respect thereto, measurement housing 13 might, for example, be formed from metal, hard resin, glass, or the like, and might have rigidity so as not to be subject to deformation. In addition, measurement housing 13 may, e.g., as is the case in the present embodiment, extend cylindrically in first direction D1, and may be arranged so as to span the exterior and the interior of case 32.

Responsive membrane 15 may, e.g., as is the case in the present embodiment, be secured to first end 13a in first direction D1 of measurement housing 13, and may be arranged in a region that is internal to cover 33 but external to case 32. As a result, because responsive membrane 15 is exposed, causing first end 10a of electrode device 10 to be immersed within target liquid X1 will cause responsive membrane 15 to come in contact with target liquid X1.

Accordingly, at measurement electrode 11, a change in the concentration of a particular water quality item (e.g., hydrogen ion or a particular ion) of target liquid X1 will cause a change in the electric potential produced by responsive membrane 15. In this way, water quality items of target liquid X1 that are detected by electrode device 10 are in accordance with the present embodiment pHs or particular ions. Note that measurement internal solution 14 is present between responsive membrane 15 and measurement internal electrode 12, and that responsive membrane 15 causes target liquid X1 and measurement internal electrode 12 to be electrically insulated from each other.

Furthermore, while there is no particular limitation with respect thereto, measurement electrode 11 may comprise a protective film that covers responsive membrane 15 and that allows particular ion(s) (e.g., hydrogen ion) of target liquid X1 to pass therethrough. In accordance with such constitution, responsive membrane 15 will be exposed by way of an intervening protective film, and will come in contact only with those substance(s) within target liquid X1 that are able to pass through the protective film.

Furthermore, measurement electrode 11 may, e.g., as is the case in the present embodiment, comprise measurement seal 17 which causes measurement internal solution 14 to be sealed in a region between it and responsive membrane 15. This will make it possible for measurement internal solution 14 to be sealed within a sealed space formed by measurement housing 13, responsive membrane 15, and measurement seal 17. While there is no particular limitation with respect thereto, note that measurement seal 17 may, for example, be formed from elastic material (e.g., rubber).

As shown in FIG. 2 through FIG. 5, reference electrode 21 comprises reference internal electrode 22; reference housing 23 within which reference internal electrode 22 is housed; reference internal solution 24 which is housed within reference housing 23 and within which reference internal electrode 22 is immersed; and liquid junction 25 which electrically connects target liquid X1 and reference internal solution 24.

In addition, at reference electrode 21, even if there is a change in the concentration of a particular water quality item (e.g., a particular ion) of target liquid X1, the electric potential produced at liquid junction 25 will not change but will remain constant. Furthermore, reference electrode 21 may, e.g., as is the case in the present embodiment, comprise lead wire(s) 26 that electrically connect reference internal electrode 22 which has electrical conductivity and terminal 34 (see FIG. 1).

While there is no particular limitation with respect thereto, liquid junction 25 may, for example, be formed from porous material (e.g., porous ceramic or the like). In addition, it is also possible, for example, to adopt a constitution in which a portion of liquid junction 25 is exposed so as to come in contact with target liquid X1, and a portion of liquid junction 25 is arranged within reference housing 23 so as to come in contact with reference internal solution 24.

More specifically, a constitution may be adopted in which, e.g., as is the case in the present embodiment, liquid junction 25 extends in first direction D1; first end 25a in first direction D1 of liquid junction 25 is exposed and is arranged at the exterior of reference housing 23; and second end 25b in first direction D1 of liquid junction 25 is arranged at the interior of reference housing 23. Note, however, that there is no limitation with respect to such constitution, it being possible, for example, for liquid junction 25 to be formed from a glass ground-joint structure.

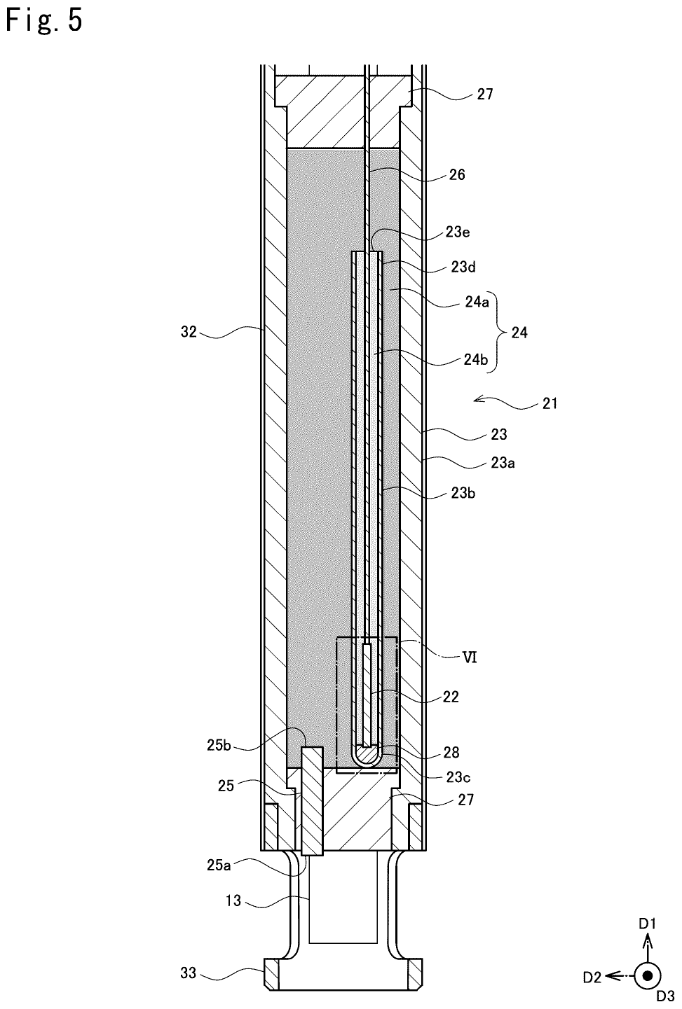

As shown in FIG. 5, reference housing 23 comprises first housing 23a at which liquid junction 25 is arranged, and second housing 23b within which reference internal electrode 22 is arranged. As a result, reference internal solution 24 comprises first internal solution 24a which is housed within first housing 23a, and second internal solution 24b which is housed within second housing 23b. While there is no particular limitation with respect thereto, first housing 23a and second housing 23b might, for example, be formed from metal, hard resin, glass, or the like, and might have rigidity so as not to be subject to deformation.

First housing 23a may, e.g., as is the case in the present embodiment, extend cylindrically in first direction D1. Furthermore, first housing 23a may, e.g., as is the case in the present embodiment, be constituted by case 32. Note, however, that there is no limitation with respect to such constitution, it being possible, for example, for first housing 23a to be a separate object that is arranged within case 32.

Second housing 23b extends cylindrically from first end 23c to second end 23d. In addition, second housing 23b comprises opening 23e at second end 23d, the interior of the foregoing second housing 23b communicating with the interior of first housing 23a only by way of opening 23e. As a result, the distance from liquid junction 25 to reference internal electrode 22 when traveling only through reference internal solution 24 will be greater than the straight-line distance between liquid junction 25 and reference internal electrode 22.

Accordingly, this will make it possible, for example, to suppress occurrence of situations in which target liquid X1 entering thereinto from liquid junction 25 might otherwise reach regions peripheral to reference internal electrode 22. As a result, because it will be possible, for example, to increase the time it takes before fluctuation in electric potential occurs due to target liquid X1 having reached a region peripheral to reference internal electrode 22, this will make it possible, for example, to increase the time during which reference electrode 21 can be used continuously.

Furthermore, a constitution may be adopted in which, e.g., as is the case in the present embodiment, second housing 23b extends linearly in first direction D1 and is arranged within first housing 23a, and in which the straight-line distance between liquid junction 25 and second end 23d that has opening 23e is greater than the straight-line distance between liquid junction 25 and first end 23c. Note, however, that there is no limitation with respect to such constitution, it being possible, for example, for second housing 23b to comprise curved portion(s); and it is furthermore possible, for example, for second housing 23b to be arranged at the exterior of first housing 23a.

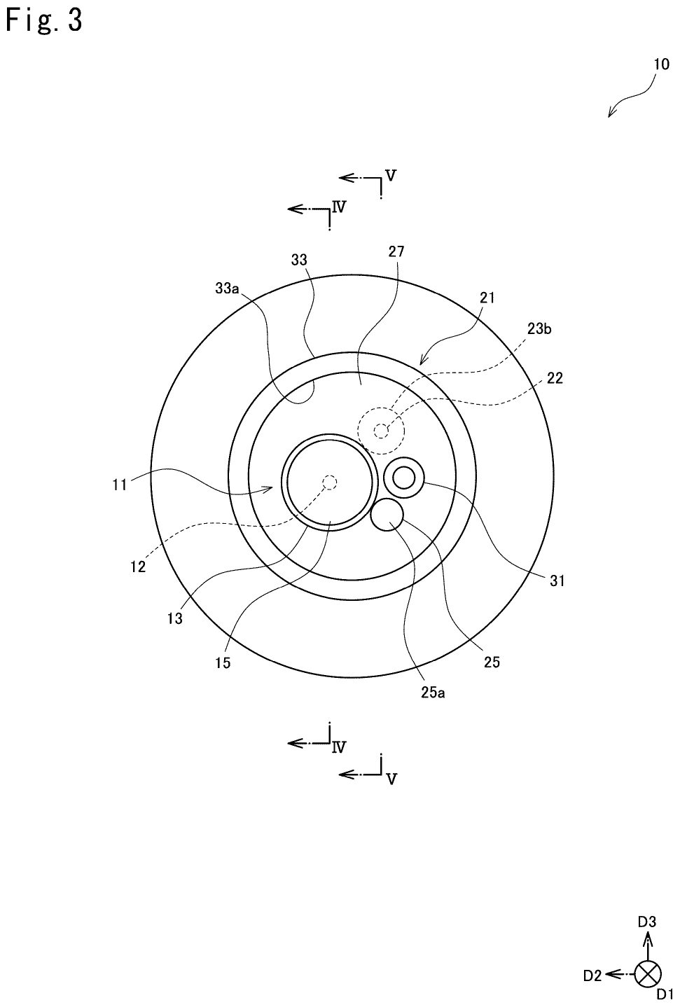

Furthermore, reference electrode 21 may, e.g., as is the case in the present embodiment, comprise reference seals 27, 27 that mutually seal reference internal solution 24 in the region therebetween. As a result, reference internal solution 24 is sealed within the sealed space formed by reference housing 23 and reference seals 27, 27. More specifically, in accordance with the present embodiment, reference internal solution 24 is sealed within the sealed space formed by first housing 23a and reference seals 27, 27 and measurement housing 13 and temperature sensor 31 (see FIG. 2 and FIG. 3). While there is no particular limitation with respect thereto, note that reference seals 27 may, for example, be formed from elastic material (e.g., rubber).

Moreover, because first internal solution 24a is a gel, second housing 23b is retained by first internal solution 24a. As a result, the location of second housing 23b relative to first housing 23a is maintained by first internal solution 24a. Accordingly, there is no need, for example, for a member such as would connect first housing 23a and second housing 23b for the purpose of causing second housing 23b to be secured to first housing 23a.

Note, however, that there are cases in which reference internal electrode 22, when immersed in reference internal solution 24 which is a liquid-like substance (gel or liquid), contains a first component that might decompose and become dissolved therewithin. While there is no particular limitation with respect thereto, where reference internal electrode 22 is, for example, formed from silver/silver chloride (Ag/AgCl), the silver chloride of reference internal electrode 22 may become dissolved within reference internal solution 24 as a result of the following decomposition reaction.

AgCl→Ag⁺+Cl⁻

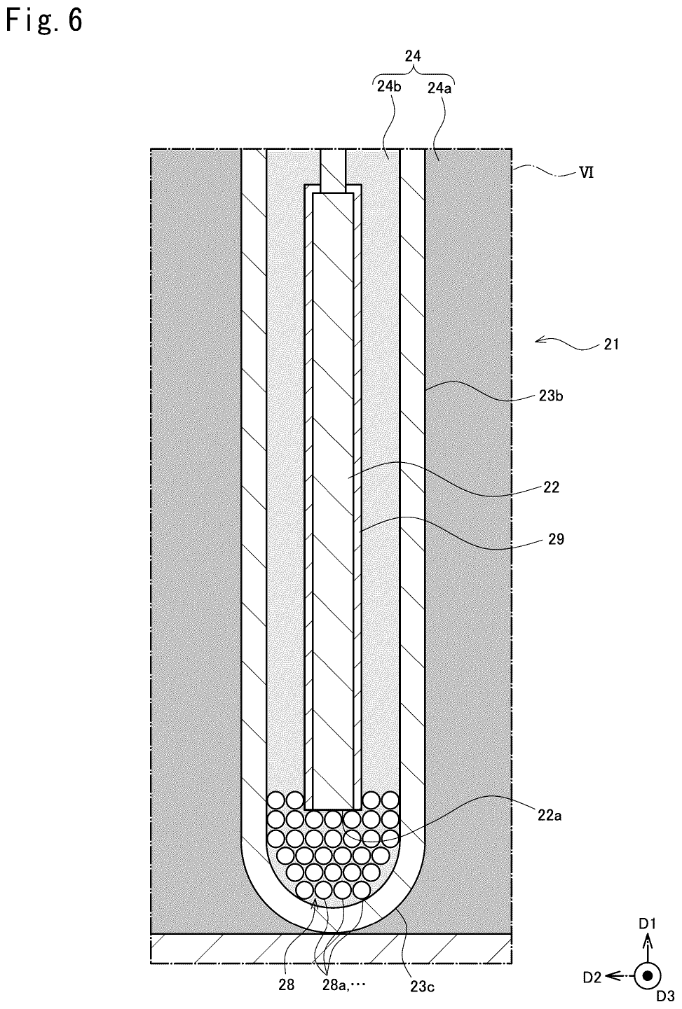

As shown in FIG. 5 and FIG. 6, reference electrode 21 therefore comprises dissolving material 28 which contains the first component and which is arranged in solid form within reference internal solution 24. While there is no particular limitation with respect thereto, in accordance with the present embodiment, reference internal electrode 22 and dissolving material 28 contain silver chloride (AgCl) which is the first component that dissolves in reference internal solution 24.

Note that the first component contained in reference internal electrode 22 and dissolving material 28 might, for example, be silver sulfide (AgS), silver bromide (AgBr), silver iodide (AgI), or silver cyanide (AgCN); or might, for example, be a combination which is any plurality chosen from among silver chloride (AgCl), silver sulfide (AgS), silver bromide (AgBr), silver iodide (AgI), and silver cyanide (AgCN).

While there is no particular limitation with respect thereto, a constitution may be adopted in which, for example, reference internal electrode 22 is formed from silver/silver chloride/silver sulfide (Ag/AgCl/AgS); and dissolving material 28 is formed from silver chloride (AgCl) and silver sulfide (AgS). That is, it is also possible to adopt a constitution in which the first component is silver chloride (AgCl) and silver sulfide (AgS). Thus, the first component might, for example, be one substance; or it might, for example, be a plurality of substances.

Furthermore, reference electrode 21 comprises covering material 29 which covers the surface of reference internal electrode 22. This will make it possible to increase the mass of the first component at reference internal electrode 22 while making it possible at the same time to reduce the surface area of reference internal electrode 22 that comes in contact with reference internal solution 24.

Covering material 29 completely covers that portion of the surface of reference internal electrode 22 which is toward second end 23d of second housing 23b from the center in first direction D1 of reference internal electrode 22. In addition, covering material 29 may, e.g., as is the case in the present embodiment, partly cover that portion of the surface of reference internal electrode 22 which is toward first end 23c of second housing 23b from the center in first direction D1 of reference internal electrode 22. This being the case, reference internal electrode 22 will come in contact with second internal solution 24b at a portion of the surface thereof which is toward first end 23c of second housing 23b from the center thereof.

While there is no particular limitation with respect thereto, a constitution may be adopted in which, e.g., as is the case in the present embodiment, reference internal electrode 22 extends in first direction D1; and covering material 29 is formed after the fashion of a cylinder that extends along the entire length in first direction D1 of reference internal electrode 22. This being the case, reference internal electrode 22 will come in contact with second internal solution 24b only at end face 22a at an end thereof in first direction D1.

Accordingly, even if reference internal electrode 22 were to become dissolved in second internal solution 24b, the area over which reference internal electrode 22 comes in contact with second internal solution 24b would remain constant (almost the same). Moreover, covering material 29 may be formed from a substance that does not react with reference internal solution 24; while there is no particular limitation with respect thereto, it might, for example, be formed from resin (e.g., Teflon or the like).

Moreover, dissolving material 28 comprises a plurality of granules 28a that are formed in granular fashion. In addition, whereas the mass of the first component at reference internal electrode 22 is greater than the mass of the first component at dissolving material 28 (more specifically, the total mass of first component in granules 28a), the surface area over which dissolving material 28 comes in contact with second internal solution 24b (more specifically, the total surface area over which granules 28a come in contact with second internal solution 24b) is greater than the surface area over which reference internal electrode 22 comes in contact with second internal solution 24b.

As a result, when, for example, the temperature of reference internal solution 24 rises and there is an increase in the solubility of the first component with respect to reference internal solution 24, this will make it possible to increase the tendency for not reference internal electrode 22 but dissolving material 28 to become dissolved in second internal solution 24b. Accordingly, because dissolving material 28 will be dissolved in second internal solution 24b, it will be possible to suppress elution of reference internal electrode 22 into second internal solution 24b. What is more, because the mass of the first component at reference internal electrode 22 is large, it will be possible to increase the life of reference internal electrode 22.

While there is no particular limitation with respect thereto, the mass % of first component at granules 28a might, for example, be not less than 50%; not less than 70% being, for example, preferred; not less than 80% being, for example, more preferred; and not less than 90% being, for example, extremely preferred. This will make it possible, for example, to ensure that there is an adequate amount of the first component at dissolving material 28.

Furthermore, the surface area of one granule 28a may, e. g., as is the case in the present embodiment, be less than the surface area over which reference internal electrode 22 comes in contact with second internal solution 24b. While there is no particular limitation with respect thereto, the diameter of granule 28a might, for example, be not greater than 2.0 mm; not greater than 1.0 mm being, for example, preferred; not greater than 0.5 mm being, for example, more preferred; and not greater than 0.1 mm being, for example, extremely preferred. This will make it possible, for example, to ensure that there is an adequate surface area over which dissolving material 28 comes in contact with second internal solution 24b (the total surface area over which granules 28a come in contact with second internal solution 24b).

Furthermore, reference internal electrode 22 and dissolving material 28 are respectively arranged within second housing 23b. More specifically, reference internal electrode 22 and dissolving material 28 are respectively arranged toward first end 23c from the center of second housing 23b.

Even more specifically, a plurality of granules 28a of dissolving material 28 have been made to accumulate at first end 23c of second housing 23b, and reference internal electrode 22 comes in contact with a plurality of granules 28a. Note that the center of second housing 23b refers to that location along the cylindrical axis extending from first end 23c to second end 23d which is at the center between first end 23c and second end 23d.

This being the case, first component from dissolving material 28 which has become dissolved therein will collect in a region that is within second housing 23b and that is in the vicinity of reference internal electrode 22. Accordingly, when the amount of first component that has already dissolved reaches the point where the solubility of the first component in that portion of second internal solution 24b which corresponds to the region in the vicinity of reference internal electrode 22 prevents it from dissolving further, further dissolving of dissolving material 28 will stop until such time as the first component is able to spread to other regions. As a result, it will be possible to suppress elution of reference internal electrode 22 into second internal solution 24b.

What is more, first component from dissolving material 28 which has become dissolved therein will collect in a location that is within second housing 23b and that is far from second end 23d. In addition, because second housing 23b communicates with first housing 23a only by way of opening 23e at second end 23d, second internal solution 24b within second housing 23b is only able to flow into first housing 23a by way of opening 23e. This makes it possible to suppress occurrence of a situation in which first component that has dissolved therein would otherwise flow out of second housing 23b. Accordingly, because it will, for example. be possible to maintain a saturated state, it will be possible to suppress further dissolving of dissolving material 28 in second internal solution 24b.

Furthermore, second internal solution 24b is a liquid-like substance which may be a gel or a liquid; it may, for example, be a gel as is the case in the present embodiment; or it may, for example, be a liquid. In addition, the jelly strength of first internal solution 24a is greater than the jelly strength of second internal solution 24b. Note that jelly strength is the value as measured in accordance with JIS K 6503 (2001).

As a result, because the jelly strength of first internal solution 24a is large, this makes it possible to suppress flow of first internal solution 24a into second housing 23b. As a result, it will be possible to suppress flow of second internal solution 24b out of second housing 23b. Accordingly, because it will be possible to suppress occurrence of a situation in which first component from dissolving material 28 which has become dissolved therein would otherwise flow out of second housing 23b, it will be possible, for example, to maintain a saturated state.

What is more, because the jelly strength of first internal solution 24a is large, this will make it possible to suppress flow of first internal solution 24a to the exterior from liquid junction 25. As a result, it will be possible to suppress flow of target liquid X1 into first housing 23a from liquid junction 25. This will make it possible to suppress admixture of target liquid X1 into reference internal solution 24 at reference housing 23 (more specifically, first internal solution 24a at first housing 23a). Accordingly, it will be possible, for example, to properly carry out measurement of water quality values of target liquid X1.

Moreover, while there is no particular limitation with respect thereto, first internal solution 24a and second internal solution 24b may, for example, respectively be acrylamide gel and gel(s) including lithium acetate, potassium chloride, and silver chloride which has been dissolved thereinto until a saturated state is reached. Furthermore, while there is no particular limitation with respect thereto, mass % of acrylamide gel at first internal solution 24a might, for example, be 150% to 300% of the mass % of acrylamide gel at second internal solution 24b.

Thus, it will be possible not only for dissolving material 28 which contains the first component to be arranged in the form of a solid within reference internal solution 24, but it will also be possible for the first component to be dissolved in reference internal solution 24. While there is no particular limitation with respect thereto, the amount of the first component which is dissolved therein might, for example, be the solubility of the first component in second internal solution 24b when at a particular temperature.

There is no particular limitation with respect to said particular temperature. For example, said particular temperature might, for example, be the temperature of reference internal solution 24 at the time of manufacture of electrode device 10. Furthermore, said particular temperature might, for example, be the air temperature at the location at which electrode device 10 will be used (e.g., maximum air temperature, average yearly air temperature, etc.).

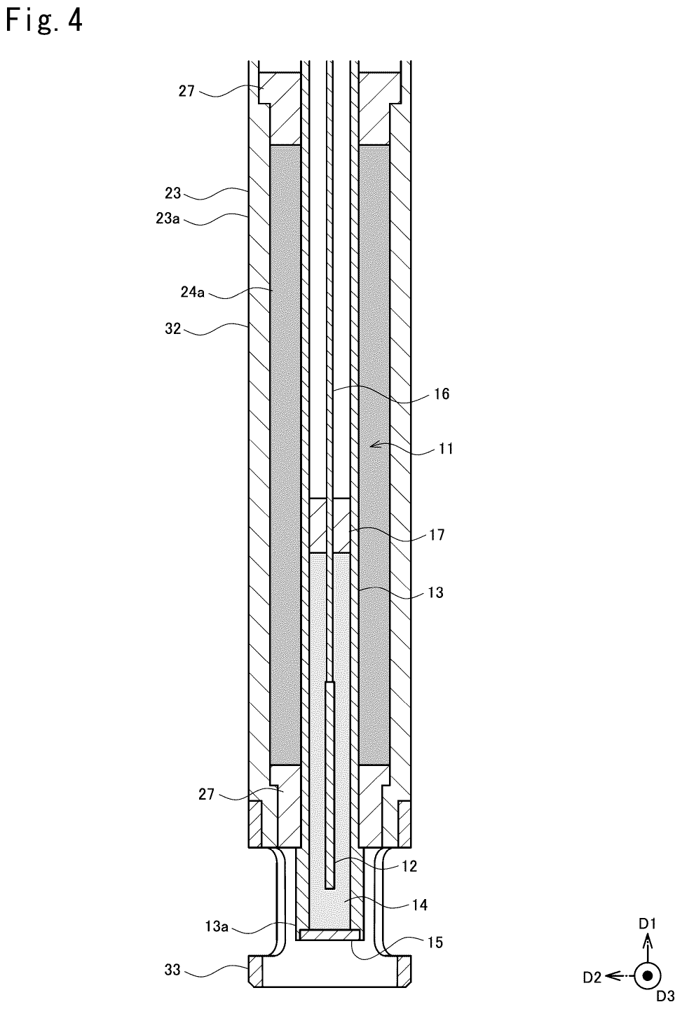

Furthermore, measurement internal solution 14 (see FIG. 4) is a liquid-like substance which may be a gel or a liquid. In addition, the jelly strength of second internal solution 24b is greater than the jelly strength of measurement internal solution 14. As a result, because the large jelly strength of second internal solution 24b will tend to cause second internal solution 24b not to flow, this will make it possible to suppress flow of second internal solution 24b out of second housing 23b. Accordingly, because it will be possible to suppress occurrence of a situation in which first component from dissolving material 28 which has become dissolved therein would otherwise flow out of second housing 23b, it will be possible, for example, to maintain a saturated state.

Moreover, while there is no particular limitation with respect thereto, measurement internal solution 14 might, for example, be a liquid that contains potassium chloride. Furthermore, while there is no particular limitation with respect thereto, measurement internal electrode 12 might, for example, be formed from silver/silver chloride.

Furthermore, those parts-other than reference internal electrode 22-which come in contact with reference internal solution 24; specifically, in accordance with the present embodiment, reference housing 23 (first housing 23a and second housing 23b), liquid junction 25, lead wire 26, reference seal 27, covering material 29, measurement housing 13, and temperature sensor 31, may, for example, be formed from substance(s) that do not react with reference internal solution 24; and furthermore, may, for example, be formed from electrically nonconductive material(s). Note, however, that lead wire 26 may, for example, be such that wire made from electrically conductive material is covered with electrically nonconductive material.

Furthermore, at electrode device 10, when the temperature of reference internal solution 24 decreases and dissolving material 28 that had been dissolved in reference internal solution 24 again becomes present therein in the form of a solid, it is acceptable for dissolving material 28 to float about within reference internal 24 at location(s) solution other than location(s) where this was present before becoming dissolved therein.

As described above, as in the present embodiment, it is preferred that
the electrode device 10 comprises a reference electrode 21,
the reference electrode 21 comprises
   a reference internal electrode 22 that contains a first component;
   a reference housing 23 within which the reference internal electrode 22 is housed;
   a reference internal solution 24 that is housed within the reference housing 23 and within which the reference internal electrode 22 is immersed; and
   a dissolving material 28 that contains the first component and that is arranged in solid form within the reference internal solution 24.

In accordance with such constitution, when the temperature of reference internal solution 24 rises and there is an increase in the solubility of the first component with respect to reference internal solution 24, because dissolving material 28 which contains the first component is arranged in the form of a solid within reference internal solution 24, dissolving material 28 will become dissolved within reference internal solution 24. As a result, because the dissolving of the dissolving material makes it possible to suppress dissolving of the first component at the reference internal electrode, this makes it possible to suppress elution of reference internal electrode into 22 reference internal solution 24.

Further, as in the present embodiment, it is preferred that the electrode device 10 includes a configuration in which:
a surface area over which the dissolving material 28 comes in contact with reference internal solution 24 is greater than a surface area over which the reference internal electrode 22 comes in contact with the reference internal solution 24.

In accordance with such constitution, because the surface area over which dissolving material 28 comes in contact with reference internal solution 24 is greater than the surface area over which reference internal electrode 22 comes in contact with reference internal solution 24, this makes it possible to cause the tendency for dissolving material 28 to become dissolved within reference internal solution 24 to be greater than the tendency for reference internal electrode 22 to become dissolved therewithin. As a result, it is possible to suppress elution of reference internal electrode 22 into reference internal solution 24.

Further, as in the present embodiment, it is preferred that the electrode device 10 includes a configuration in which:
a mass of the first component at the reference internal electrode 22 is greater than a mass of the first component at the dissolving material 28; and
the reference electrode 21 comprises a covering material 29 that covers a surface of the reference internal electrode 22.

In accordance with such constitution, because covering material 29 covers the surface of reference internal electrode 22, it is possible to cause the surface area over which dissolving material 28 comes in contact with reference internal solution 24 to be greater than the surface area over which reference internal electrode 22 comes in contact with reference internal solution 24. What is more, because the mass of the first component at reference internal electrode 22 is greater than the mass of the first component at dissolving material 28, it is possible to increase the life of reference internal electrode 22.

Further, as in the present embodiment, it is preferred that the electrode device 10 includes a configuration in which:
the reference electrode 21 comprises a liquid junction 25 that electrically connects a target liquid X1 and the reference internal solution 24; and
the reference housing 23 comprises
   a first housing 23a at which the liquid junction 25 is arranged; and
   a second housing 23b within which the reference internal electrode 22 and the dissolving material 28 are respectively arranged;
wherein the second housing 23b extends cylindrically from a first end 23c to a second end 23d; and
the second housing 23b comprises an opening 23e at the second end 23d, and communicates with the first housing 23a only by way of the opening 23e.

In accordance with such constitution, because reference internal electrode 22 and dissolving material 28 are respectively arranged within second housing 23b, first component from dissolving material 28 which has become dissolved therein will collect in a region that is within second housing 23b. As a result, when the amount of first component that has already dissolved reaches the point where the solubility of the first component in the internal solution (the second internal solution in accordance with the present embodiment) 24b at second housing 23b prevents it from dissolving further, further dissolving of dissolving material 28 will stop. Accordingly, it will be possible to suppress elution of reference internal electrode 22 into second internal solution 24b at second housing 23b.

What is more, second housing 23b communicates with first housing 23a only by way of opening 23e at second end 23d. As a result, because second internal solution 24b within second housing 23b is only able to flow into first housing 23a by way of opening 23e, it will be possible to suppress occurrence of a situation in which first component of dissolving material 28 that has dissolved therein would otherwise flow out of second housing 23b.

Further, as in the present embodiment, it is preferred that the electrode device 10 includes a configuration in which:
the reference internal electrode 22 and the dissolving material 28 are respectively arranged toward the first end 23c from a center of the second housing 23b.

In accordance with such constitution, first component from dissolving material 28 which has become dissolved therein will collect in a region that is in the vicinity of reference internal electrode 22 and that is toward first end 23c from the center of second housing 23b. As a result, when the amount of first component that has already dissolved reaches the point where the solubility of the first component in that portion of the internal solution (the second internal solution in accordance with the present embodiment) 24b at second housing 23b which corresponds to the region in the vicinity of reference internal electrode 22 prevents it from dissolving further, further dissolving of dissolving material 28 will stop until such time as the first component is able to spread to other regions. Accordingly, it will be possible to suppress elution of reference internal electrode 22 into second internal solution 24b at second housing 23b.

What is more, first component from dissolving material 28 which has become dissolved therein will collect at a location that is toward first end 23c from the center of second housing 23b; i.e., a location that is far from opening 23e at second end 23d of second housing 23b. This will make it possible to suppress occurrence of a situation in which first component of dissolving material 28 that has dissolved therein would otherwise flow out of opening 23e at second end 23d of second housing 23b.

Further, as in the present embodiment, it is preferred that the electrode device 10 includes a configuration in which:
the dissolving material 28 is arranged at the first end 23c of the second housing 23b;
the reference electrode 21 comprises a covering material 29 that covers a surface of the reference internal electrode 22; and
the covering material 29 completely covers that portion of the surface of the reference internal electrode 22 which is toward the second end 23d of the second housing 23b from a center of the reference internal electrode 22.

In accordance with such constitution, because dissolving material 28 is arranged at first end 23c of second housing 23b, it is possible to cause first component from dissolving material 28 that has become dissolved therein to collect at a location that is far from opening 23e at second end 23d of second housing 23b. This will make it possible to suppress occurrence of a situation in which first component of dissolving material 28 that has dissolved therein would otherwise flow out of opening 23e at second end 23d of second housing 23b.

In addition, because covering material 29 completely covers that portion of the surface of reference internal electrode 22 which is toward second end 23d of second housing 23b from the center of reference internal electrode 22, reference internal electrode 22 will come in contact with internal solution (the second internal solution in accordance with the present embodiment) 24b at second housing 23b in a region which is toward first end 23c of second housing 23b from the center thereof. This being the case, first component from dissolving material 28 that has become dissolved therein will collect in a region in the vicinity of that portion of reference internal electrode 22 which corresponds to the region at which it comes in contact with second internal solution 24b at second housing 23b.

Accordingly, when the amount of first component that has already dissolved reaches the point where the solubility of the first component in that portion of second internal solution 24b at second housing 23b which corresponds to the region in the vicinity of said region at reference internal electrode 22 prevents it from dissolving further, further dissolving of dissolving material 28 will stop until such time as the first component is able to spread to other regions. As a result, it will be possible to suppress elution of reference internal electrode 22 into internal solution 24b at second housing 23b.

Further, as in the present embodiment, it is preferred that the electrode device 10 includes a configuration in which:
a portion of a surface of the reference internal electrode 22 comes in contact with a portion of a surface of the dissolving material 28.

In accordance with such constitution, because a portion of the surface of reference internal electrode 22 will come in contact with a portion of the surface of dissolving material 28, first component from dissolving material 28 that has become dissolved therein will collect in the vicinity of reference internal electrode 22. As a result, when the amount of first component that has already dissolved reaches the point where the solubility of the first component in that portion of second internal solution 24b at second housing 23b which corresponds to the region in the vicinity of reference internal electrode 22 prevents it from dissolving further, further dissolving of dissolving material 28 will stop until such time as the first component is able to spread to other regions. Accordingly, it will be possible to suppress elution of reference internal electrode 22 into second internal solution 24b at second housing 23b.

Further, as in the present embodiment, it is preferred that the electrode device 10 includes a configuration in which:
the dissolving material 28 comprises a plurality of granules 28a that are formed so as to be granular; and
the plurality of granules 28a has been made to accumulate at the first end 23c of the second housing 23b.

In accordance with such constitution, because dissolving material 28 comprises a plurality of granules 28a which contain the first component, this will increase the tendency for second internal solution 24b to come in contact with the entirety of granules 28a. This will make it possible to increase the tendency for dissolving material 28 to become dissolved in internal solution (the second internal solution in accordance with the present embodiment) 24b. Accordingly, it will be possible to suppress elution of reference internal electrode 22 into reference internal solution 24.

Further, as in the present embodiment, it is preferred that the electrode device 10 includes a configuration in which:
the reference internal solution 24 comprises
   a first internal solution 24a that is housed within the first housing 23a; and
   a second internal solution 24b that is housed within the second housing 23b; and
a jelly strength of the first internal solution 24a is greater than a jelly strength of the second internal solution 24b.

In accordance with such constitution, because the jelly strength of first internal solution 24a is greater than the jelly strength of second internal solution 24b, this makes it possible to suppress flow of first internal solution 24a into second housing 23b. As a result, it will be possible to suppress flow of second internal solution 24b out of second housing 23b. This will make it possible to suppress occurrence of a situation in which first component of dissolving material 28 that has dissolved therein would otherwise flow out of second housing 23b.

What is more, because the jelly strength of first internal solution 24a is greater than the jelly strength of second internal solution 24b, this will make it possible to suppress flow of first internal solution 24a to the exterior from liquid junction 25. As a result, it will be possible to suppress flow of target liquid X1 into first housing 23a from liquid junction 25. This will make it possible to suppress admixture of target liquid X1 into reference internal solution 24 at reference housing 23.

Further, as in the present embodiment, it is preferred that the electrode device 10 includes a configuration in which:
the reference internal solution 24 comprises
   a first internal solution 24a that is housed within the first housing 23a; and
   a second internal solution 24b that is housed within the second housing 23b;
the first internal solution 24a is a gel; and
a location of the second housing 23b relative to the first housing 23a is maintained by the first internal solution 24a.

In accordance with such constitution, because first internal solution 24a is a gel, second housing 23b will be retained by first internal solution 24a. As a result, the location of second housing 23b relative to first housing 23a will be maintained.

Further, as in the present embodiment, it is preferred that the electrode device 10 comprises
a measurement electrode 11 for measuring a water quality value of a target liquid X1 based on a difference in electric potential between the measurement electrode 11 and the reference electrode 21.

In accordance with such constitution, because reference electrode 21 and measurement electrode 11 are comprised thereby, electrode device 10 is a composite electrode. This will make it possible to measure water quality value(s) of target liquid X1 based on difference(s) in electric potential between measurement electrode 11 and reference electrode 21.

Further, as in the present embodiment, it is preferred that the electrode device 10 further comprises
a measurement electrode 11 for measuring a water quality value of a target liquid X1 based on a difference in electric potential between the measurement electrode 11 and the reference electrode 21;
wherein the measurement electrode 11 comprises
   a measurement internal electrode 12;
   a measurement housing 13 within which the measurement internal electrode 12 is housed; and
   a measurement internal solution 14 that is housed within the measurement housing 13 and within which the measurement internal electrode 12 is immersed; and
the jelly strength of the second internal solution 24b is greater than a jelly strength of the measurement internal solution 14.

In accordance with such constitution, because the fact that the jelly strength of second internal solution 24b is greater than the jelly strength of measurement internal solution 14 tends to cause second internal solution 24b not to flow, this makes it possible to suppress flow of second internal solution 24b out of second housing 23b. This will make it possible to suppress occurrence of a situation in which first component of dissolving material 28 that has dissolved therein would otherwise flow out of second housing 23b.

Further, as in the present embodiment, it is preferred that
the water quality measuring device 1 comprises the electrode device 10.

In accordance with such constitution, it will be possible to suppress elution of reference internal electrode 22 into reference internal solution 24.

Further, as in the present embodiment, it is preferred that
the water quality measuring method wherein the electrode device 10 is used to measure a water quality value of a target liquid X1.

In accordance with such method, it will be possible to suppress elution of reference internal electrode 22 into reference internal solution 24.

The electrode device 1, the water quality measuring device 10 and the water quality measuring method are not limited to the configuration of the embodiment described above, and the effects are not limited to those described above. It goes without saying that the electrode device 1, the water quality measuring device 10 and the water quality measuring method can be variously modified without departing from the scope of the subject matter of the present invention. For example, the constituents, methods, and the like of various modified examples described below may be arbitrarily selected and employed as the constituents, methods, and the like of the embodiments described above, as a matter of course.

(A) The constitution of electrode device 10 associated with the foregoing embodiment is such that the surface area over which dissolving material 28 comes in contact with reference internal solution 24 is greater than the surface area over which reference internal electrode 22 comes in contact with reference internal solution 24. However, electrode device 10 is not limited to such constitution.

For example, it is also possible to adopt a constitution in which the surface area over which dissolving material 28 comes in contact with reference internal solution 24 is less than the surface area over which reference internal electrode 22 comes in contact with reference internal solution 24. Furthermore, it is also possible, for example, to adopt a constitution in which the surface area over which dissolving material 28 comes in contact with reference internal solution 24 is the same as the surface area over which reference internal electrode 22 comes in contact with reference internal solution 24.

(B) Furthermore, the constitution of electrode device 10 associated with the foregoing embodiment is such that the mass of the first component at reference internal electrode 22 is greater than the mass of the first component at dissolving material 28. However, electrode device 10 is not limited to such constitution.

For example, it is also possible to adopt a constitution in which the mass of the first component at reference internal electrode 22 is less than the mass of the first component at dissolving material 28. Furthermore, it is also possible, for example, to adopt a constitution in which the mass of the first component at reference internal electrode 22 is the same as the mass of the first component at dissolving material 28.

(C) Furthermore, the constitution of electrode device 10 associated with the foregoing embodiment is such that reference electrode 21 comprises covering material 29 which covers the surface of reference internal electrode 22. However, electrode device 10 is not limited to such constitution. For example, it is also possible to adopt a constitution in which reference electrode 21 does not comprise covering material 29, and the surface area of reference electrode 21 that comes in contact with reference internal solution 24 is the same as the surface area of reference electrode 21.

(D) Furthermore, the constitution of electrode device 10 associated with the foregoing embodiment is such that covering material 29 completely covers that portion of the surface of reference internal electrode 22 which is toward second end 23d of second housing 23b from the center of reference internal electrode 22; more specifically, the constitution is such that reference internal electrode 22 comes in contact with reference internal solution 24 at a portion of the surface thereof which is toward first end 23c of second housing 23b from the center thereof. However, electrode device 10 is not limited to such constitution.

For example, it is also possible to adopt a constitution in which covering material 29 partly covers that portion of the surface of reference internal electrode 22 which is toward second end 23d of second housing 23b from the center of reference internal electrode 22. In addition, it is also possible, for example, to adopt a constitution in which reference internal electrode 22 comes in contact with reference internal solution 24 at a portion of the surface thereof which is toward second end 23d of second housing 23b from the center thereof.

Furthermore, it is also possible, for example, to adopt a constitution in which covering material 29 does not completely cover that portion of the surface of reference internal electrode 22 which is toward second end 23d of second housing 23b from the center of reference internal electrode 22, but covers at least a portion of the surface thereof which is toward first end 23c of second housing 23b from the center of reference internal electrode 22. In addition, it is also possible, for example, to adopt a constitution in which reference internal electrode 22 comes in contact with reference internal solution 24 over the entire portion of the surface thereof which is toward second end 23d of second housing 23b from the center thereof.

Furthermore, it is also possible, for example, to adopt a constitution in which covering material 29 does not completely cover that portion of the surface of reference internal electrode 22 which is toward first end 23c of second housing 23b from the center of reference internal electrode 22, but covers at least a portion of the surface thereof which is toward second end 23d of second housing 23b from the center of reference internal electrode 22. In addition, it is also possible, for example, to adopt a constitution in which reference internal electrode 22 comes in contact with reference internal solution 24 over the entire portion of the surface thereof which is toward first end 23c of second housing 23b from the center thereof.

(E) Furthermore, the constitution of electrode device 10 associated with the foregoing embodiment is such that reference housing 23 comprises first housing 23a and second housing 23b into which the interior thereof is partitioned. However, electrode device 10 is not limited to such constitution. For example, it is also possible to adopt a constitution in which reference housing 23 is not partitioned at the interior thereof.

(F) Furthermore, the constitution of electrode device 10 associated with the foregoing embodiment is such that first internal solution 24a is a gel, and second housing 23b is such that the location thereof relative to first housing 23a is maintained by first internal solution 24a. However, electrode device 10 is not limited to such constitution.

For example, it is also possible to adopt a constitution in which first internal solution 24a is a liquid. Furthermore, it is also possible, for example, to adopt a constitution in which reference electrode 21 comprises a member that connects first housing 23a and second housing 23b for the purpose of causing second housing 23b to be secured to first housing 23a.

(G) Furthermore, the constitution of electrode device 10 associated with the foregoing embodiment is such that the jelly strength of first internal solution 24a is greater than the jelly strength of second internal solution 24b. However, electrode device 10 is not limited to such constitution.

For example, it is also possible to adopt a constitution in which the jelly strength of first internal solution 24a is the same as the jelly strength of second internal solution 24b. In the context of such a constitution, it is also possible, for example, to adopt a constitution in which first internal solution 24a and second internal solution 24b are a common internal solution (i.e., the internal solutions have the same components and the same concentrations). Furthermore, it is also possible, for example, to adopt a constitution in which the jelly strength of first internal solution 24a is less than the jelly strength of second internal solution 24b.

(H) Furthermore, the constitution of electrode device 10 associated with the foregoing embodiment is such that the jelly strength of second internal solution 24b is greater than the jelly strength of measurement internal solution 14. However, electrode device 10 is not limited to such constitution.

For example, it is also possible to adopt a constitution in which the jelly strength of second internal solution 24b is less than the jelly strength of measurement internal solution 14. Furthermore, it is also possible, for example, to adopt a constitution in which the jelly strength of second internal solution 24b is the same as the jelly strength of measurement internal solution 14.

(I) Furthermore, the constitution of electrode device 10 associated with the foregoing embodiment is such that reference internal electrode 22 and dissolving material 28 are respectively arranged toward first end 23c from the center of second housing 23b. However, electrode device 10 is not limited to such constitution.

For example, it is also possible to adopt a constitution in which at least one of reference internal electrode 22 or dissolving material 28 is arranged toward second end 23d from the center of second housing 23b. Furthermore, it is also possible, for example, to adopt a constitution in which at least one of reference internal electrode 22 or dissolving material 28 is arranged within first housing 23a.

(J) Furthermore, the constitution of electrode device 10 associated with the foregoing embodiment is such that a portion of the surface of reference internal electrode 22 comes in contact with a portion of the surface of dissolving material 28; more specifically, a portion of first end face 22a of reference internal electrode 22 comes in contact with a portion of plurality of granules 28a. However, electrode device 10 is not limited to such constitution. For example, it is also possible to adopt a constitution in which the surface of reference internal electrode 22 is separated from dissolving material 28 (all of granules 28a).

(K) Furthermore, the constitution of electrode device 10 associated with the foregoing embodiment is such that dissolving material 28 comprises a plurality of granules 28a that contain the first component. However, electrode device 10 is not limited to such constitution.

For example, it is also possible to adopt a constitution in which dissolving material 28 is made up of a single solid that contains the first component. In addition, while there is no particular limitation with respect thereto, in the context of such constitution, it is also possible, for example, to adopt a constitution in which the surface area over which dissolving material 28 comes in contact with reference internal solution 24 is greater than the surface area over which reference internal electrode 22 comes in contact with reference internal solution 24. Furthermore, while there is no particular limitation with respect thereto, in the context of such constitution, it is also possible, for example, to adopt a constitution in which the mass of the first component at reference internal electrode 22 is greater than the mass of the first component at dissolving material 28.

(L) Furthermore, the constitution of electrode device 10 associated with the foregoing embodiment is such that dissolving material 28 is arranged at first end 23c of second housing 23b; more specifically, a plurality of granules 28a have been made to accumulate at first end 23c of second housing 23b. However, electrode device 10 is not limited to such constitution.

For example, it is also possible to adopt a constitution in which dissolving material 28 is arranged at location(s) other than first end 23c of second housing 23b. Furthermore, it is also possible, for example, to adopt a constitution in which a plurality of granules 28a have been made to accumulate at location(s) other than first end 23c of second housing 23b.

(M) Furthermore, the constitution of electrode device 10 associated with the foregoing embodiment is such that it comprises measurement electrode 11 for measuring water quality value(s) of target liquid X1 based on difference(s) in electric potential between it and reference electrode 21. However, electrode device 10 is not limited to such constitution. For example, it is also possible to adopt a constitution in which electrode device 10 comprises only reference electrode 21 and does not comprise measurement electrode 11.

(N) Furthermore, the constitution of electrode device 10 associated with the foregoing embodiment is such that water quality items of target liquid X1 that are detected thereby are pHs or particular ions; that is, measurement electrode 11 comprises measurement internal solution 14 and responsive membrane 15. However, electrode device 10 is not limited to such constitution.

For example, a water quality item of target liquid X1 that is detected by electrode device 10 may be an oxidation-reduction potential (ORP). In addition, in the context of such a situation, it is also possible to adopt a constitution in which, for example, measurement electrode 11 is such that measurement internal electrode 12 is arranged so as to be exposed (e.g., by causing measurement internal electrode 12 to be secured to the tip of measurement housing 13) so as to cause measurement internal electrode 12 to come in contact with target liquid X1, and in which measurement electrode 11 does not comprise either measurement internal solution 14 or responsive membrane 15.

(O) Note that, except where the results of a previous operation is used in a subsequent operation, the order of execution of actions, procedures, steps, stages, and other such respective operations taking place, for example, in the context of the method(s) and apparatus(es) indicated in the claims, specification, and drawings may be implemented in any desired order. Notwithstanding that words such as “first,” “next,” and so forth may have been employed for convenience of description, this should not be understood to imply that execution must be carried out in that order.

1. An electrode device comprising a reference electrode,
the reference electrode comprising
a reference internal electrode that contains a first component;
a reference housing within which the reference internal electrode is housed;
a reference internal solution that is housed within the reference housing and within which the reference internal electrode is immersed; and
a dissolving material that contains the first component and that is arranged in solid form within the reference internal solution.

2. The electrode device according to claim 1 wherein
a surface area over which the dissolving material comes in contact with reference internal solution is greater than a surface area over which the reference internal electrode comes in contact with the reference internal solution.

3. The electrode device according to claim 2 wherein
a mass of the first component at the reference internal electrode is greater than a mass of the first component at the dissolving material; and
the reference electrode comprises a covering material that covers a surface of the reference internal electrode.

4. The electrode device according to claim 1 wherein
the reference electrode comprises a liquid junction that electrically connects a target liquid and the reference internal solution; and
the reference housing comprises
a first housing at which the liquid junction is arranged; and
a second housing within which the reference internal electrode and the dissolving material are respectively arranged;
wherein the second housing extends cylindrically from a first end to a second end; and
the second housing comprises an opening at the second end, and communicates with the first housing only by way of the opening.

5. The electrode device according to claim 4 wherein
the reference internal electrode and the dissolving material are respectively arranged toward the first end from a center of the second housing.

6. The electrode device according to claim 5 wherein
the dissolving material is arranged at the first end of the second housing;
the reference electrode comprises a covering material that covers a surface of the reference internal electrode; and
the covering material completely covers that portion of the surface of the reference internal electrode which is toward the second end of the second housing from a center of the reference internal electrode.

7. The electrode device according to claim 5 wherein
a portion of a surface of the reference internal electrode comes in contact with a portion of a surface of the dissolving material.

8. The electrode device according to claim 4 wherein
the dissolving material comprises a plurality of granules that are formed so as to be granular; and
the plurality of granules has been made to accumulate at the first end of the second housing.

9. The electrode device according to claim 4 wherein
the reference internal solution comprises
a first internal solution that is housed within the first housing; and
a second internal solution that is housed within the second housing; and
a jelly strength of the first internal solution is greater than a jelly strength of the second internal solution.

10. The electrode device according to claim 4 wherein
the reference internal solution comprises a first internal solution that is housed within
the first housing; and
a second internal solution that is housed within the second housing;
the first internal solution is a gel; and
a location of the second housing relative to the first housing is maintained by the first internal solution.

11. The electrode device according to claim 1 further comprising
measurement electrode for measuring a water quality value of a target liquid based on a difference in electric potential between the measurement electrode and the reference electrode.

12. The electrode device according to claim 9 further comprising
measurement electrode for measuring a water quality value of a target liquid based on a difference in electric potential between the measurement electrode and the reference electrode;
wherein the measurement electrode comprises
a measurement internal electrode;
a measurement housing within which the measurement internal electrode is housed; and
a measurement internal solution that is housed within the measurement housing and within which the measurement internal electrode is immersed; and
the jelly strength of the second internal solution is greater than a jelly strength of the measurement internal solution.

13. The electrode device according to claim 3 wherein
the reference internal electrode extends in a first direction;
the covering material extends cylindrically along an entire length in the first direction of the reference internal electrode; and
the reference internal electrode comes in contact with the reference internal solution only at an end face at an end of the reference internal electrode in the first direction.

14. The electrode device according to claim 8 wherein
a mass of the first component at the reference internal electrode is greater than a mass of the first component at the dissolving material;
the reference electrode comprises a covering material that covers a surface of the reference internal electrode; and
a total surface area over which the plurality of granules come in contact with the reference internal solution is greater than a surface area over which the reference internal electrode comes in contact with the reference internal solution.

15. The electrode device according to claim 8 wherein
a portion of a surface of the reference internal electrode comes in contact with at least one granule among the plurality of granules.

16. The electrode device according to claim 8 wherein
a surface area of at least one among the plurality of granules is less than a surface area over which the reference internal electrode comes in contact with the reference internal solution.

17. The electrode device according to claim 16 wherein
a total surface area over which the plurality of granules come in contact with the reference internal solution is greater than a surface area over which the reference internal electrode comes in contact with the reference internal solution.

18. The electrode device according to claim 1 wherein
the reference internal solution contains the first component.

19. A water quality measuring device comprising the electrode device according to claim 1.

20. A water quality measuring method wherein the electrode device according to claim 1 is used to measure a water quality value of a target liquid.
